(19) United States
(12) Patent Application Publication
(10) Pub. No.: US 2012/0107319 A1
(43) Pub. Date: May 3, 2012

(54) Pharmaceutical compositions comprising antibodies binding to the intracellular domain of EBV (Epstein-Barr virus) latent membrane protein-1 (LMP1)

(75) Inventor: Tadamasa Ooka, Lyon (FR)

(73) Assignees: Centre National De La Recherche Scientifique(CNRS), Paris (FR); UNIVERSITE CLAUDE BERNAND LYON 1, Villeurbanne (FR)

(21) Appl. No.: 13/057,736

(22) PCT Filed: Aug. 7, 2009
(86) PCT No.: PCT/EP2009/060277

§ 371 Date: Apr. 29, 2011

(30) Foreign Application Priority Data
Aug. 8, 2008 (FR) 08162083.3

(51) Int. Cl. A61K 39/395; A61K 38/08; A61K 31/7088; C07K 7/06; A61P 31/12; A61P 35/00; C07K 16/08; C07H 21/04; A61K 38/16; A61K 38/10; C12N 5/10; C07K 7/08

(52) U.S. Cl. 424/139.1; 435/366; 514/3.7; 514/44 R; 530/327; 530/328; 530/329; 530/330; 530/387.9; 536/23.72

(57) ABSTRACT

The invention relates to pharmaceutical and vaccine compositions comprising an antibody binding specifically to the intracellular domain of EBV protein LMP1.

The present invention relates to polypeptide fragments derived from the intracellular domain of LMP-1 and to antibodies specifically binding these fragments, to their uses in immunotherapy and vaccination.

The Epstein-Barr virus (EBV) is associated with several human cancers: Nasopharyngeal carcinoma, Gastric carcinoma, Burkitt's lymphoma, Hodgkin's lymphoma, lymphoma induced in AIDS patients, Esophage and Intrahepatic cholangiocarcinoma. Recent data showed that EBV is also implicated in nasal NK/T-cell lymphoma and intra-hepatic cholangiocarcinoma. Oral hairy leucoplasia (OHL), frequent in AIDS patients is also tigtly associated with EBV. EBV is therefore both lymphotropic and epitheliotropic.

Several therapeutic methods for EBV-related cancers have been used including radio- and chemo-therapy. However radio- and chemotherapy pose classical problems (toxicity, dose, etc.). Several cellular and viral gene therapies have also been developed which are generally based on viral and/or cellular proteins as targets. However, these therapies have not performed sufficiently well.

In immunotherapy, anti-EGFR antibodies (Epidermal Growth Factor Receptor) were also proposed, particularly for treatment of carcinomas (NPC, Thymomes, Lung, Cervical carcinoma, Colon, Breast, and Head and Neck), because epithelial tumor cells associated or not with EBV express EGFR. The treatment is therefore not exclusive for EBV-associated carcinomas. Efficiency of the treatment (monoclonal antibody Cetumximab) is being evaluated for cervical cancer and thymoma. However, there is a risk that patients treated with anti-EGFR in combination with radiotherapy become radio-resistant.

Nasopharyngeal carcinoma (NPC) is a human malignancy derived from the epithelium of the retro-nasal cavity. It is one of the most striking examples of a human malignancy that is consistently associated with a virus. The full-length genome of Epstein-Barr Virus (EBV) is contained in all malignant NPC cells and it encodes viral proteins that probably contribute to the malignant phenotype (Decaussin G, Sbih-Lammali F, De Turenne-Tessier M, Bougermouh A M, Ooka T. 2000. Cancer Res 60: 5584-5588; Ooka T: 2005. In. Epstein-Barr Virus. Horizon Press, Annette Griffin: Edited by Erle S. Robertson. Chapter 28: p.p 613-630). Even though EBV infection is ubiquitous in humans, the incidence of NPC is extremely variable depending on the geographic area. About 5-10% of gastric carcinomas in the world are also associated with EBV.

NPC biopsies expressed consistently several EBV genes in including genes encoding EBERs, EBNA1, LMP1, LMP2A, BARF0 and BARF1. Among them, only LMP1 and BARF-1 are capable of inducing malignant transformation in rodent fibroblasts (Wei and Ooka, 1989, EMBO J. 8:2897-903; Wang D, Liebowitz D and Kieff E. 1985. Cell 43:831-840) and are considered as viral oncogenes.

LMP1 (Latent membrane protein-1) belongs to a family of latent antigens expressed on the surface of cells infected by EBV and indispensable for B cell immortalization. LMP1 is encoded by the genome of the Epstein-Barr Virus belonging to Human Herpesvirus 4 type 1. LMP1 possesses six transmembrane domains and an intracellular C-terminal domain. The C-terminal region includes two major functional domains, CTAR1, and CTAR2. The extracellular domains called <<short loops>> of LMP1 protein are present on the surface of EBV-infected cells. LMP1 is essential for B cell immortalisation activating several cellular genes, like NFkB, A20 and EGF-R which can inhibit cell differentiation when transfected into epithelial cells (Ooka T: 2005. In. Epstein-Barr Virus. Horizon Press, Annette Griffin: Edited by Erle S. Robertson. Chapter 28: p.p 613-630.). However, LMP1 alone is unable to immortalise B cells and it needs to collaborate with five other EBV genes (EBERs, LMP2A, EBNA3A, EBNA3B, EBNA2) (Kieff and Rickinson, 2007, Fields Virology 5th Edition-Fields B N, Knipe D M, Howley P M (ed.) Lippincott-Williams & Wilkins Publishers: Philadelphia, 2007, pp. 2603-2654).

Classically, LMP1 proteins have been localized on the cellular membrane. However recent data showed that LMP1 could be secreted and localized in exosomal components in the culture medium of B95-8 cells (non human marmosette B lymphocyte), as well as in the culture medium of insect Sf9 cells infected with LMP1 recombinant Baculovirus (Vazirabadi G, Geiger T R, Coffin W F, Martin J M. Links 2003, J Gen Virol. 84: 1997-2008; Flanagan J, Middeldorp J, Sculley, T. 2003, J Gen Virol 84:1871-9) and in the culture medium of NPC-derived c666-1 cell line (Houali K, X. Wang, Y. Shimizu, D. Djennaoui, J. Nicholls, S. Fiorini, A. Bougermouh and T. Ooka. Clin. Cancer Res. 2007 13: 4993-5000). These exosomal components are likely responsible for the inhibition of T cell proliferation (Flanagan J, Middeldorp J, Sculley, T. 2003 J Gen Virol 84:1871-9). LMP-1 present within exosome-like vesicles has been shown to activate FGF2 expression (Ceccarelli S, Visco V, Raffa S, Wakisaka N, Pagano J, Torrissi R. 2007 Int. J. Cancer 121: 1494-506).

The essential oncogenic role of LMP1 is determined by its activation of NFkB. The inhibition of LMP1 expression resulted in cell apoptosis linked to the diminution of NFkB expression (Kieff and Rickinson, 2007, Fields Virology 5th Edition-Fields B N, Knipe D M, Howley P M (ed.) Lippincott-Williams & Wilkins Publishers: Philadelphia, 2007, pp. 2603-2654).

The secretion of two oncoproteins (LMP1 and BARF1) in serum and saliva of NPC patients was recently demonstrated (Houali K, X. Wang, Y. Shimizu, D. Djennaoui, J. Nicholls, S. Fiorini, A. Bougermouh and T. Ooka. Clin. Cancer Res. 2007. 13: 4993-5000) and these oncoproteins purified from serum of NPC patient showed powerful mitogenic activity in vitro. This mitogenic activity could be related to the development of tumors.

A majority of the LMP1 found in serum of NPC patient or in serum of mouse developing NPC-derived tumor induced after injection of c666-1 cells is associated with exosome-like vesicules. This complexed form, LMP1/exosome, is able to activate cell cycle by an autocrine mechanism, while free LMP1 (without exosome) is unable to activate the cell cycle (Houali K, X. Wang, Y. Shimizu, D. Djennaoui, J. Nicholls, S. Fiorini, A. Bougermouh and T. Ooka. Clin. Cancer Res. 2007. 13: 4993-5000).

U.S. Pat. No. 6,723,695 describes CTL epitopes within EBV structural and latent proteins. These CTL epitopes could be effective in providing antiviral immunity against EBV infection. Clinical trials have been initiated for the treatment of EBV-positive lymphoma. Epitopes derived from LMP1 are derived from the extracellular loops of LMP1.

In immunotherapy, EBV-specific CTLs which recognize LMP1 epitopes were used also for treatment of Hodgkin disease patients. However, the treatment was not successful due to the inhibitory effect by cytokines (Gottschalk et al., 2002, Adv. Cancer Res. 8: 175-201; Bollard et al., 2004. J. Exp. Med. 200: 1623-1633).

WO03/048337 describes antibodies binding to LMP1 and their uses in therapeutic methods. The anti-LMP1 antibodies bind to the extracellular loops of LMP1 which are exposed on the surface of infected cells. Inhibition of cell growth observed with these antibodies is not clearly detailed and is probably due to the neutralisation of LMP1 localized on cellular membrane and not due to binding of LMP1 localized on exosomes secreted into the culture medium.

EP-A-1 229 043 describes different peptides derived from LMP1 and antibody reagents reactive therewith. The polypeptides and antibodies described may be used for the preparation of a medicament for the treatment of EBV infection or EBV positive tumors. Antibodies against the intracellular deomain of LMP1 are described. However, pharmaceutical compositions are only envisioned with antibodies raised against the extracellular loops of LMP1.

The role of LMP1 as an oncogene required for the immortalization of B cells has been described. However, other oncogenes have been described and are required for immortalization.

In the state of the art, immunotherapy has been directed against the extracellular loops of LMP1 which are exposed on the surface of EBV infected cells.

The present invention proposes new immunotherapy methods based on the functional inhibition of LMP1. Surprisingly, the inhibition of LMP 1 function is sufficient to prevent and suppress tumor development.

The present invention unexpectedly shows that antibodies binding to the intracellular domain of LMP1 are sufficient both in vitro and in vivo to inhibit the development of tumor cells associated with EBV. Antibodies binding the intracellular domain of LMP1 are capable of neutralising the oncoprotein in vivo resulting in the prevention and suppression of tumors in a mouse model. This neutralisation could be due to the fact that the intracellular domain of LMP1 is exposed on the surface of exosomes.

A monoclonal anti-LMP1 antibody commercialized by BD. Sciences, France was used. This antibody binds to the intracellular domain of LMP1 between the CTRA1 and CTAR2 domains of LMP1. Successive injection of anti-LMP1 antibody before injection of NPC-derived epithelial tumor cells led to prevention of tumor apparition. When anti-LMP1 was successively injected after the tumor size became about 0.8 cm in diameter, the tumor regressed and completely disappeared. This represents the first report on immunotherapy with anti-LMP1 antibodies suppressing and protecting from EBV positive tumors.

Addition of anti-LMP1 into culture medium was also able to inhibit EBV-positive B cell growth, suggesting that immunotherapy based on anti LMP1 is also efficient to inhibit and protect from the development of EBV-associated lymphomas.

Further, immunotherapy targeting the intracellular domain of LMP 1 is promising for prevention and treatment of NPC, because patients show very low antibody responses to this viral protein (Meij P, Vervoort M B H J, Aarbiou J, van Dissel P, Brink A, Bloemena E, Meijer C J L M, Middeldorp J M. 1999. J. Infect. Diseases 179: 1108-15).

### Sequence Listing

SEQ ID No. 1: Amino acid sequence of LMP1(Latent Membrane Protein-1) from human Herpesvirus 4 type 1 (Genbank YP_{—}401722.1)

### DESCRIPTION OF THE INVENTION

A first object of the present invention is a composition for use as a medicament comprising an antibody or an antibody fragment binding specifically to the polypeptide derived from Epstein-Barr Virus protein LMP1 having the sequence from position 188 to position 386 of SEQ ID No. 1.

In a preferred embodiment, the composition for use as a medicament comprises an antibody or an antibody fragment binding specifically to a fragment of at least 5, 7, 10, 15, 20, 50 amino acids of the polypeptide derived from Epstein-Barr Virus protein LMP1 having the sequence from position 188 to position 386 of SEQ ID No. 1.

Preferably, the composition for use as a medicament comprises an antibody or an antibody fragment binding specifically to the polypeptide derived from Epstein-Barr Virus protein LMP1 having the sequence from position 232 to position 351 of SEQ ID No. 1.

Even more preferred, the composition for use as medicament comprises an antibody or antibody fragment binding specifically to the polypeptide derived from Epstein-Barr Virus protein LMP1 having the sequence from position 306 to position 318 of SEQ ID No. 1.

A second object of the present invention is a composition for use as a medicament or as a vaccine comprising a fragment of at least 10, 20, 50 amino acids of the polypeptide derived from Epstein-Barr Virus protein LMP1 having the sequence from position 188 to position 386 of SEQ ID No. 1.

In a preferred embodiment, the composition for use as a medicament or as a vaccine according comprises the polypeptide derived from Epstein-Barr Virus protein LMP1 having the sequence from position 188 to position 386 of SEQ ID No. 1.

Preferably, the composition for use as a medicament or as a vaccine comprises the polypeptide derived from Epstein-Barr Virus protein LMP1 having the sequence from position 232 to position 351 of SEQ ID No. 1.

In another preferred embodiment, the composition for use as a medicament or as a vaccine comprises the polypeptide derived from Epstein-Barr Virus protein LMP1 having the sequence from position 306 to position 318 of SEQ ID No. 1.

Another object of the present invention is a composition for use as a medicament or as a vaccine comprising a polynucleotide encoding a polypeptide selected from the group consisting of: a fragment of at least 5, 7, 10, 15, 20, 50 amino acids of the polypeptide derived from Epstein-Barr Virus protein LMP1 having the sequence from position 188 to position 386 of SEQ ID No. 1, the polypeptide derived from Epstein-Barr Virus protein LMP1 having the sequence from position 188 to position 386 of SEQ ID No. 1 or the polypeptide derived from Epstein-Barr Virus protein LMP1 having the sequence from position 306 to position 318 of SEQ ID No. 1.

The present invention encompasses pharmaceutical compositions and vaccine compositions.

Preferably, the compositions of the present invention are for prevention or treatment of EBV positive tumors or EBV associated tumors.

More preferably, the compositions of the present invention are for prevention or treatment of nasopharyngeal carcinoma, gastric carcinoma, Burkitt's lymphoma, Hodgkin's lymphoma, lymphoma induced in AIDS patients, esophage and intrahepatic cholangiocarcinoma, nasal NK/T-cell lymphoma and oral hairy leucoplasia (OHL).

Even more preferably, the compositions of the present invention are for prevention or treatment of nasopharyngeal carcinoma.

Another object of the present invention is a peptide derived from Epstein-Barr Virus protein LMP 1 selected from the group consisting of:
the peptide having the sequence from position 306 to position 318 of SEQ ID No. 1,
a fragment of at least 5, 7 or 10 amino acids of the peptide having the sequence from position 306 to position 318 of SEQ ID No. 1.

Another objet of the present invention is a polynucleotide encoding a peptide according to the invention.

The invention further relates to a host cell transformed with a polynucleotide according to the invention.

The present invention relates to compositions for use as a medicament comprising an antibody or antibody fragment binding specifically to the intracellular fragment of LMP1 or a derivative thereof as described herein. The present invention further relates to compositions for use as a medicament or as a vaccine comprising the intracellular domain of LMP1 or a fragment thereof. Another object of the present invention is a composition for use as a medicament or as a vaccine comprising a polynucleotide encoding the intracellular domain of LMP1 or a fragment thereof.

The polypeptide having the sequence from position 188 to position 386 of SEQ ID No. 1 corresponds to the intracellular domain of LMP1 which is not exposed on the surface of EBV infected cells. However, it has been surprisingly found in the present invention that antibodies binding to this domain prevent and reduce tumor development in an in vivo mouse model.

The present invention provides pharmaceutical compositions comprising:
a) an effective amount of an antibody or antibody fragment as described herein, an effective amount of a polypeptide as described herein or an effective amount of a polynucleotide as described herein, and
b) a pharmaceutically acceptable carrier, which may be inert or physiologically active.

The present invention further provides vaccine compositions comprising:
a) an effective amount of a polypeptide as described herein or an effective amount of a polynucleotide as described herein, and
b) an adjuvant.

As used herein, “pharmaceutically-acceptable carriers” includes any and all solvents, dispersion media, coatings, antibacterial and antifungal agents, and the like that are physiologically compatible. Examples of suitable carriers, diluents and/or excipients include one or more of water, saline, phosphate buffered saline, dextrose, glycerol, ethanol, and the like, as well as combination thereof. In many cases, it will be preferable to include isotonic agents, such as sugars, polyalcohols, or sodium chloride in the composition. In particular, relevant examples of suitable carrier include: (1) Dulbecco's phosphate buffered saline, pH˜7.4, containing or not containing about 1 mg/ml to 25 mg/ml human serum albumin, (2) 0.9% saline (0.9% w/v sodium chloride (NaCl)), and (3) 5% (w/v) dextrose; and may also contain an antioxidant such as tryptamine and a stabilizing agent such as Tween 20.

The pharmaceutical compositions encompassed by the present invention may also contain a further therapeutic agent for the treatment of cancers associated With EBV.

The compositions of the invention may be in a variety of forms. These include for example liquid, semi-solid, and solid dosage forms, but the preferred form depends on the intended mode of administration and therapeutic application. Typical preferred compositions are in the form of injectable or infusible solutions. The preferred mode of administration is parenteral (e.g. intravenous, intramuscular, intraperinoneal, subcutaneous). In a preferred embodiment, the compositions of the invention are administered intravenously as a bolus or by continuous infusion over a period of time. In another preferred embodiment, they are injected by intramuscular, subcutaneous, intra-articular, intrasynovial, intratumoral, peritumoral, intralesional, or perilesional routes, to exert local as well as systemic therapeutic effects.

Sterile compositions for parenteral administration can be prepared by incorporating the antibody, the antibody fragment, the polypeptide, or the polynucleotide as described in the present invention in the required amount in the appropriate solvent, followed by sterilization by microfiltration. As solvent or vehicle, there may be used water, saline, phosphate buffered saline, dextrose, glycerol, ethanol, and the like, as well as combination thereof. In many cases, it will be preferable to include isotonic agents, such as sugars, polyalcohols, or sodium chloride in the composition. These compositions may also contain adjuvants, in particular wetting, isotonizing, emulsifying, dispersing and stabilizing agents. Sterile compositions for parenteral administration may also be prepared in the form of sterile solid compositions which may be dissolved at the time of use in sterile water or any other injectable sterile medium.

The antibody, antibody fragment, polypeptide or polynucleotide as described herein may also be orally administered. As solid compositions for oral administration, tablets, pills, powders (gelatine capsules, sachets) or granules may be used. In these compositions, the active ingredient according to the invention is mixed with one or more inert diluents, such as starch, cellulose, sucrose, lactose or silica, under an argon stream. These compositions may also comprise substances other than diluents, for example one or more lubricants such as magnesium stearate or talc, a coloring, a coating (sugar-coated tablet) or a glaze.

As liquid compositions for oral administration, there may be used pharmaceutically acceptable solutions, suspensions, emulsions, syrups and elixirs containing inert diluents such as water, ethanol, glycerol, vegetable oils or paraffin oil. These compositions may comprise substances other than diluents, for example wetting, sweetening, thickening, flavoring or stabilizing products.

The doses depend on the desired effect, the duration of the treatment and the route of administration used.

The invention is also related to the use of an antibody, antibody fragment, polypeptide or polynucleotide as described herein for the manufacture of a medicament or for the manufacture of a vaccine for the prevention or treatment of EBV positive tumors or EBV associated tumors such as nasopharyngeal carcinoma, gastric carcinoma, Burkitt's lymphoma, Hodgkin's lymphoma, lymphoma induced in AIDS patients, esophage and intrahepatic cholangiocarcinoma, nasal NK/T-cell lymphoma and oral hairy leucoplasia (OHL).

In a preferred embodiment, antibodies, antibody fragments, polypeptides or polynucleotides as described herein, are used for prevention or treatment of EBV positive tumors. In a more preferred embodiment, one of the pharmaceutical or vaccine compositions disclosed above, and which contains an antibody, antibody fragment, polypeptide or polynucleotide as described herein, is used for prevention or treatment of EBV positive tumors. More preferably, they are used for prevention or treatment of nasopharyngeal carcinoma, gastric carcinoma, Burkitt's lymphoma, Hodgkin's lymphoma, lymphoma induced in AIDS patients, esophage and intrahepatic cholangiocarcinoma, nasal NK/T-cell lymphoma and oral hairy leucoplasia (OHL). In a preferred embodiment, they are used for prevention or treatment of nasopharyngeal carcinoma.

The present invention also provides methods for preventing or treating EBV positive tumors including administering an effective amount of an antibody, antibody fragment, polypeptide or polynucleotide as described herein to a human or to a patient in need thereof. In a preferred embodiment, the invention relates to methods for prevention or treatment of nasopharyngeal carcinoma, gastric carcinoma, Burkitt's lymphoma, Hodgkin's lymphoma, lymphoma induced in AIDS patients, esophage and intrahepatic cholangiocarcinoma, nasal NK/T-cell lymphoma and oral hairy leucoplasia (OHL). Even more preferred, the invention relates to methods for prevention or treatment of nasopharyngeal carcinoma.

In a first embodiment, the compositions of the present invention comprise an antibody or an antibody fragment binding specifically to the intracellular domain of LMP1 or a derivative thereof.

As used herein the term “binding” refers to an antibody or antibody fragment that reacts with an epitope of the intracellular domain of LMP1 corresponding to the polypeptide from position 188 to position 386 of SEQ ID No. 1 or that was raised against the intracellular domain of LMP1 corresponding to the polypeptide from position 188 to position 386 of SEQ ID No. 1. Preferably, the antibody reacts with an epitope from the peptide from position 306 to 318 of SEQ ID No. 1 or was raised against the peptide from position 306 to 318 of SEQ ID No. 1. Preferably, the antibody binds specifically to the intracellular domain of LMP1 and does not crossreact with other antigens. Thus, the antibody reacts with one specific antigen.

Antibodies binding specifically to the intracellular domain of LMP1 are available commercially such as for example antibody S12 available from BD Sciences (France). Alternatively, antibodies binding specifically to the intracellular domain of LMP1 or to fragments thereof, may be produced by standard techniques. Preferred antibodies are antibodies binding to the peptide having the sequence from position 306 to 318 of SEQ ID No. 1 which is also specifically bound by monoclonal antibody S12. Preferably, the antibodies bind to the same epitope as antibody S12. The epitope of antibody S12 may be determined according to methods known to the skilled person starting from the peptide described herein having the sequence from position 306 to 318 of SEQ ID No. 1.

The term “antibody” is used herein in the broadest sense and specifically covers monoclonal antibodies of any isotype such as IgG, IgM, IgA, IgD and IgE, polyclonal antibodies, chimeric antibodies, humanized antibodies and antibody fragments. An antibody reactive with a specific antigen can be generated by recombinant methods such as selection of libraries of recombinant antibodies in phage or similar vectors, or by immunizing an animal with the antigen or an antigen-encoding nucleic acid.

A typical IgG antibody is comprised of two identical heavy chains and two identical light chains that are joined by disulfide bonds. Each heavy and light chain contains a constant region and a variable region. Each variable region contains three segments called “complementarity-determining regions” (“CDRs”) or “hypervariable regions”, which are primarily responsible for binding an epitope of an antigen. They are usually referred to as CDR1, CDR2, and CDR3, numbered sequentially from the N-terminus. The more highly conserved portions of the variable regions are called the “framework regions”.

As used herein, “VH” or “VH” refers to the variable region of an immunoglobulin heavy chain of an antibody, including the heavy chain of an Fv, scFv, dsFv, Fab, Fab′ or F(ab′)2 fragment. Reference to “VL” or “VL” refers to the variable region of the immunoglobulin light chain of an antibody, including the light chain of an Fv, scFv, dsFv, Fab, Fab′ or F(ab′)2 fragment.

A “polyclonal antibody” is an antibody which was produced among or in the presence of one or more other, non-identical antibodies. In general, polyclonal antibodies are produced from a B-lymphocyte in the presence of several other B-lymphocytes producing non-identical antibodies. Usually, polyclonal antibodies are obtained directly from an immunized animal.

A “monoclonal antibody”, as used herein, is an antibody obtained from a population of substantially homogeneous antibodies, i.e. the antibodies forming this population are essentially identical except for possible naturally occurring mutations which might be present in minor amounts. These antibodies are directed against a single epitope and are therefore highly specific.

An “epitope” is the site on the antigen to which an antibody binds. As used herein, a “chimeric antibody” is an antibody in which the constant region, or a portion thereof, is altered, replaced, or exchanged, so that the variable region is linked to a constant region of a different species, or belonging to another antibody class or subclass. “Chimeric antibody” also refers to an antibody in which the variable region, or a portion thereof, is altered, replaced, or exchanged, so that the constant region is linked to a variable region of a different species, or belonging to another antibody class or subclass. Methods for producing chimeric antibodies are known in the art.

The term “humanized antibody”, as used herein, refers to a chimeric antibody which contain minimal sequence derived from non-human immunoglobulin. The goal of humanization is a reduction in the immunogenicity of a xenogenic antibody, such as a murine antibody, for introduction into a human, while maintaining the full antigen binding affinity and specificity of the antibody. Humanized antibodies, or antibodies adapted for non-rejection by other mammals, may be produced using several technologies such as resurfacing and CDR grafting. Humanized chimeric antibodies preferably have constant regions and variable regions other than the complementarity determining regions (CDRs) derived substantially or exclusively from the corresponding human antibody regions and CDRs derived substantially or exclusively from a mammal other than a human.

The antibodies of the present invention include both the full length antibodies discussed above, as well as epitope-binding fragments thereof. As used herein, “antibody fragments” include any portion of an antibody that retains the ability to bind to the epitope recognized by the full length antibody, generally termed “epitope-binding fragments.” Examples of antibody fragments include, but are not limited to, Fab, Fab′ and F(ab′)2, Fd, single-chain Fvs (scFv), single-chain antibodies, disulfide-linked Fvs (dsFv) and fragments comprising either a VL or VH region. Epitope-binding fragments, including single-chain antibodies, may comprise the variable region(s) alone or in combination with the entirety or a portion of the following: hinge region, CH1, CH2, and CH3 domains.

In a second embodiment, the compositions of the present invention comprise a polypeptide corresponding to the intracellular domain of LMP1 or a fragment thereof.

The term polypeptide “fragments” refers to a polypeptide including part but not all of the polypeptide from which it is derived. The fragments according to this invention retain the antigenic properties of the polypeptides from which they are derived. The invention thus relates to a fragment of at least 5, 7, 10, 15, 20 amino acids of the polypeptide having the sequence from position 188 to position 386 of SEQ ID No. 1.

Advantageously, the fragments according to the invention have a minimal size while retaining their antigenic properties.

Another object of the present invention is a peptide derived from Epstein-Barr Virus protein LMP1 selected in the group consisting of:
the peptide having the sequence from position 306 to position 318 of SEQ ID No. 1,
a fragment of at least 5, 7 or 10 amino acids of the peptide having the sequence from position 306 to position 318 of SEQ ID No. 1.

In a third embodiment, the compositions of the present invention comprise a polynucleotide encoding a polypeptide as described above corresponding to the intracellular domain of LMP1 or a fragment thereof.

The term “polynucleotide” according to the present invention refers to a single strand nucleotide chain or its complementary strand which can be of the DNA or RNA type, or a double strand nucleotide chain which can be of the cDNA (complementary) or genomic DNA type. Preferably, the polynucleotides of the invention are of the DNA type, namely double strand DNA. The term “polynucleotide” also refers to modified polynucleotides.

The polynucleotides of this invention are isolated or purified from their natural environment. Preferably, the polynucleotides of this invention can be prepared using conventional molecular biology techniques such as those described by Sambrook et al. (Molecular Cloning: A Laboratory Manual, 1989) or by chemical synthesis.

Another object of the invention is a polynucleotide encoding a peptide as described herein.

The invention also relates to host cells transformed with a polynucleotide according to the invention. The man skilled in the art is well aware of the standard methods for incorporation of a polynucleotide into a host cell, for example transfection, lipofection, electroporation, microinjection, viral infection, thermal shock, transformation after chemical permeabilisation of the membrane or cell fusion.

Another object of the present invention is a vector comprising a polynucleotide according to the invention including a viral vector.

In a fourth embodiment, the compositions of the present invention comprise a transformed host cell expressing a polypeptide as described above corresponding to the intracellular domain of LMP1 or a fragment thereof.

### FIGURES

FIG. 1: Structure of LMP 1 protein and recognition site of S12 on exosome/LMP 1 complex.

FIG. 2: Effect of anti-LMP1 on EBV positive or EBV negative cell lines

Effect of anti-LMP1 was analysed on EBV-positive and EBV-negative B cell lines, and on the c666-1 epithelial cell line. Survival of the cells was monitored for 120 hours after addition of 5 μg of monoclonal antibody S12. Anti-LMP1 inhibited efficiently cell growth of c666-1, Raji and IB4, while no inhibitory effect on EBV-negative Louckes cell line was observed.

FIG. 3: MTT test for CEM (human T cell), EBV-negative AKATA (B cell), Balb/c3T3 (rodent fibroblast) and HaCaT (human epithelial cell) treated with exosome/LMP1 isolated from serum of NPC patients

Exosome/LMP1 complex (ELC) was isolated. MTT test was carried out with 50000 cells/100 μl of culture medium without FBS with 5 μA of exosome/LMP1 complex containing 300 ng of complex from NPC patient (SNPC). With or without FBS and exosome isolated from healthy individuals (EC-SNP) were used as controls. Louckes and AKATA: human B cell lines, CEM, Balb/c3T3 and HaCaT. Addition of monoclonal antibody S12 in the exosome/LMP1 assay abolished almost totally the mitogenic activity (ELC+S12).

FIG. 4: Effect of monoclonal antibody S12 on EBV-AGS cell growth

EBV-negative AGS (1) and EBV-positive AGS (2) were tested by S12 antibody. Five μg of monoclonal S12 was added in culture medium. Control cells did not receive antibody. Cell viability was measured by coommassi blue staining during 5 days.

FIG. 5: Immunotherapy assays

Anti-LMP 1 S12 was injected before (b), simultaneously (c) or after injection of c666-1 (d) cells. 50 μg of antibody were injected intrapenetorially. 10⁷ cells (c666-1) were injected subcutaneously. The values presented in the figure correspond to the average tumor size diameter measured in mm. Protocol 1: (b) with S12 for c666-1: Protocol 2: (c) with S12 for c666-1. Protocol 3: (d) with S12 for c666-1. Tumor development after injection of c666-1 cells without any antibody (a).

FIG. 6: Immunotherapy assays

Anti-LMP1 S12 was injected before (b), simultaneously (c) or after injection of EBV-AGS (d) cells. 50 μg of antibody were injected intrapenetorially. 10⁷ cells (EBV-AGS) were injected subcutaneously. The values presented in the figure correspond to the average tumor size diameter measured in mm. Protocol 1: (b) with S12 for EBV-AGS: Protocol 2: (c) with S12 for EBV-AGS. Protocol 3: (d) with S12 for EBV-AGS. Tumor development after injection of AGS-EBV cells without any antibody (a).

FIG. 7: Effect of anti-EBV DNAase

50 μg of rabbit polyclonal Anti-EBV DNAase was used for treatment every 5 days during 20 days, then 10⁶ c666-1 cells were injected. Tumor development was monitored. No inhibitory effect of the antibody on tumor development.

FIG. 8: Effect of anti-rabbit or anti-mouse

50 μg of anti-mouse Ig were treated every 5 days during 20 days, then 10⁶ c666-1 cells were injected. Tumor development was monitored. No inhibitory effect of the antidody on tumor development.

FIG. 9: Detection of LMP1/exosome complex in mouse serum and tumor cells by immunoblot LMP1/exosome complex was isolated and analysed on 12% SDS-polyacrylamide gel. Antigen antibody complexes were detected by an enhanced chemiluminescence system (ECL; Amersham). The presence of LMP1 was analyzed in serum from mice developing c666-1 or EBV-AGS tumor (1). Positive control was P3HR1 cell. LMP1/exosome complex isolated from serum: (2) S-c666-1. LMP1/exosome complex isolated from tumor (3): MT-c666-1. S12 was revealed by secondary rabbit anti-Ig). Commercial mouse Ig was used as positive control: Ig (1,2,3).

FIG. 10: Exosome/LMP1/S12 complex

Exosome/LMP 1/S12 complex was purified from mouse serum dvelopping c666-1 tumor and treated with anti-mouse Ig (for detection of S12) or anti-CD63 (for detection of exosome). Detection of exosome/LMP1/S12 complex by 10 nm glod-labeled mouse Ig and by 5 nm gold-labeled anti-CD63. Normal exosome: exosome/LMP1/S12 not-treated by these antibodies. Immunological specificity was controlled by the omission of primary antibodies or their replacement by non-immune serum.

FIG. 11

A: Translational expression of NF-kB in c666-1, AGS, EBV-AGS, c666-1, c666-1 tumor and EBV-AGS tumor.

a: Expression of five components of NF-kB (p65,p50,p52,RelB and c-Rel) was analysed by ELISA test (TransAM NFkB family kit: Ref. 43296, Active-Motif, Belgium). AGS, EBV-AGS, EBV-AGS+S12, EBV-AGS Tumor, c666-1, c666-1+S12, c666-1 tumor, Raji and S12-treated Raji were subjected to analyze expression of five components of NF-kB. The p65 and p50, majors components of NF-kB, were activated in Raji and these components were significantly inhibited by the presence of S12. Expression of the components was activated in tumor, while cells in culture showed a basal expression of the components.

b: Activation of these components were observed when Louckes cell was treated in vitro with LMP1/exosome complex isolated from NPC serum (Louckes+ELC) (b). This activation was totally reduced by the presence of S12 antibody suggesting that the activation was due to the presence of LMP1 complexed with exosome (Louckes+ELC+S12). As positive control, significant expression of p65 and p50 in Raji cells (Raji) was also totally inhibited by S12 antibody (Raji+S12).

### EXAMPLES

### Treatment with Anti-LMP-1 for Tumor Suppression

We show that treatment with anti-LMP-1 antibody 1) suppressed NPC-derived and GC-derived tumor and 2) protected from the development of NPC-derived and GC-derived tumor.

To demonstrate anti-LMP-1 antibody as a protective and suppressive agent of EBV-associated carcinomas (NPC and GC), we used an animal model (nude mice) developped previously in our laboratory (Houali K, X. Wang, Y. Shimizu, D. Djennaoui, J. Nicholls, S. Fiorini, A. Bougermouh and T. Ooka. Clin. Cancer Res. 13: 4993-5000; Sheng W, Decaussin, G., Sumner, S. and Ooka, T. 2001. Oncogene 20: 1176-1185).

Nude mice used here come from Harlan (France) produced in Italy: Strain: Hsd: Athymic Nude-Fox1^{nu}. We also tested HsdCpb:NMRI-Fox1^{nu}. Their age is 4 weeks. Their sex is male. Their weight at 4 weeks is about 19-21 g.

For in vitro analysis on the effect of anti-LMP-1 antibody, monoclonal anti-LMP-1 S12 was examined in EBV-positive NPC-derived c666-1 and GC-derived EBV-AGS epithelial cell lines and EBV-positive or EBV-negative human B cell lines.

Anti-LMP1 antibody S12 is commercialized by BD Sciences (France). Catalog number: 559898.

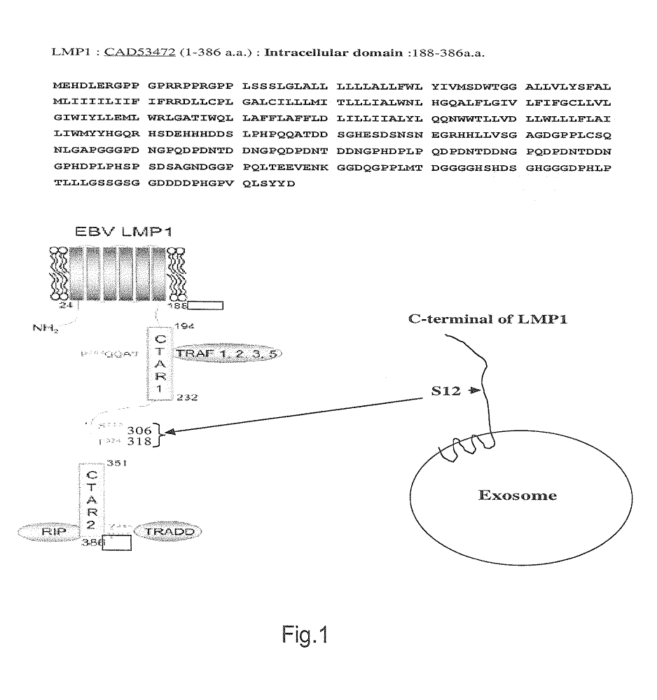

This antibody recognizes the C-terminal region of LMP1 protein, position 301-318 a.a. near CTAR 2 (see FIG. 1).

NPC-derived tumor could be induced when NPC-derived c666-1 (Cheung S T, Huang D P, Hui A B, Lo K W, Ko C W, Tsang Y S, Wong N, Whitney B M, Lee J C. Int J Cancer 1999; 83:121-6) or GC-derived EBV-positive AGS (Kassis J, Maeda A, Teramoto N, Takada, K, Wu C, Wells A. Int. J. Cancer 2002; 99: 644-51) epithelial cells were injected in nude mice. We then analyzed the effect of anti-LMP1 antibodics in these mice.

In general, AGS cells without EBV genome do not induce any tumor when injected in nude mice, but the development of GC-derived tumor occurred with EBV-positive AGS in nude mice. This observation had never been done before.

### In Vitro Experiment

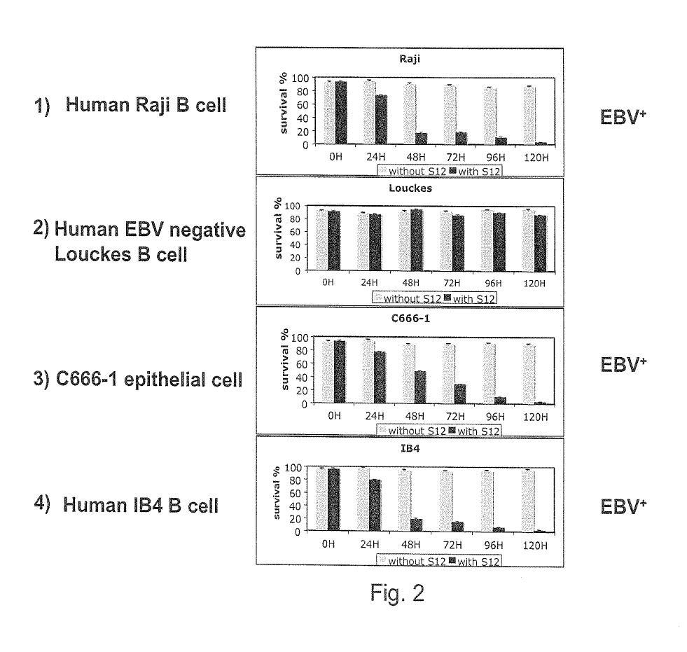

At first, the effect of anti LMP1 antibody (added in culture medium) was analysed on EBV-positive c666-1 and EBV-positive AGS epitheial cell lines, EBV-positive human IB4 B cell line, EBV-positive human Raji B cell line and EBV-negative human Louckes B cell line in culture in vitro. 5 μg of anti-LMP1 for 10⁵ cells was added in culture medium. The evolution of cell growth was observed during 120 hours (FIG. 2).

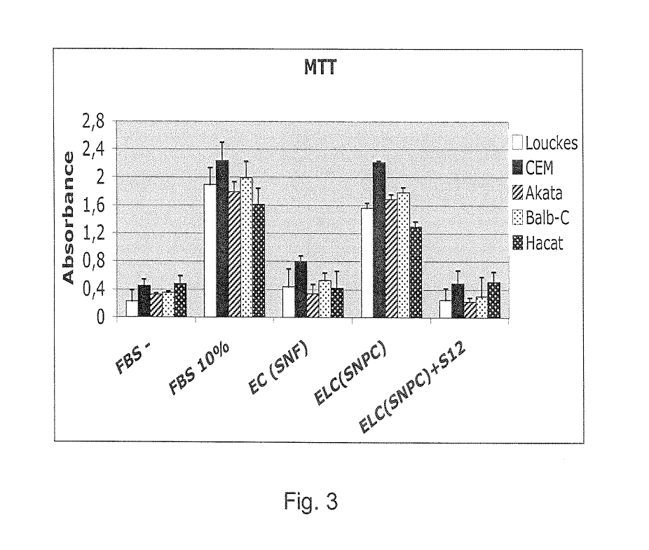

When the secreted LMP-1 oncoprotein was neutralised by 5 μg of S12 anti-LMP-1 antibody (added in culture medium), the c666-1 cells went to die as presented by survival curve in the FIG. 2-3. After addition of antibody, the survival cells diminished to 78% after 24 hours, 50% after 48 hours, 25% after 72 hours, 7% after 96 hours and all c666-1 cells went to die after 120 hours (5 days). This suggests that mitogenic activity of LMP-1/exosome is directly related to main cell activation process (Houali K, X. Wang, Y. Shimizu, D. Djennaoui, J. Nicholls, S. Fiorini, A. Bougermouh and T. Ooka. Clin. Cancer Res. 13: 4993-5000).

Similar inhibitory effect was observed in EBV-positive human Raji (FIG. 1-1) and IB4 B cell lines (FIG. 2-4). The inhibitory effect was drastic in both EBV-positive B cell lines (FIGS. 2-1 and 4): after addition of antibody, the survival cells diminished to 75% after 24 hours, 13% after 48 hours, 10% after 72 hours, 5-7% after 96 hours and all B cells went to die after 120 hours (5 days).

No such inhibitory effect ²was observed on EBV-negative Louckes B cell line) (FIG. 2-2).

In conclusion, anti-LMP-1 antibody could inhibit cell growth of EBV-positive c666-1 epithelial cell and EBV-positive B cells expressing LMP-1 protein.

These results indicate that anti-LMP-1 complexes with LMP-1/exosome secreted from cells, then the complex could enter into cell. Probably once the complex entering in cells triggered cell death in inhibiting NFkB expression (see in vivo experiment section and FIG. 15).

To verify the hypothesis, Raji cells were cultured with 5 μg S12 during 96 hours in the same condition as FIG. 2-1 (Human Raji B cell). Every 24 hours, the cells were collected, deposited onto slide and fixed with aceton to permeabilize. The presence of exosome/LMP-1/S12 complex in cell was searched with anti-mouse Ig-coupled with fluoscein.

No fluorescence was observed in Raji untreated with S12 (FIG. 2, Raji+Mouse Ig), while at 24 hours after S12-treated Raji showed a patched immunofluorescence near the cell membrane. At 96 hours, important immunofluorescence was observed at cytoplasmic and nuclear fractions. This suggests that exosome/LMP-1 secreted from tumor cell coud complex with S12, then the complex of LMP1/exosoe/S12 could be absorbed into cell and reached to nuclei. Negative response obtained in S12-untreated cells indicates that antibody S12 alone was not absorbed into cell.

We then verified if similar phenomenon (absorption of LMP1/exosome complex into cell) could be also happened when LMP1/exosome complex (ELC) was directly added in culture medium of EBV-negative cell lines. For this, we first purified the complex of exosome/LMP-1 from serum of NPC patient, then directly added in the culture medium of EBV-negative cells. Human T cell line, CEM1 and human B cell line, Louckes were cultured with 1 μg of ELC. The cells were fixed, then permeabilized. The presence of exosome/LMP-1 complex was searched by confocal microscopy using anti-LMP-1 S12 and anti-CD63 (specific marker of exosome) during 24 hours. To localize the nucleus, the cells were stained with Dapi. The incubation was carried out with the first antibody S12 or anti-CD63 with a dilution of 1/1000, followed by incubation with Alexa fluo 488 IgG goat anti-mouse IgG as a secondary antibody. Red fluorescence with rodamin for LMP1 and green fluorescence with fluoscein for CD63. The cells were excited at 356 nm (Dapi) and 488 nm (Alexa).

Both antibodies (anti-LMP-1 and anti-CD63) were co-localized in cellular compartment: cytoplasm and nuclei. These suggest that the complex of exosome/LMP-1/S12 could be absorbed as previously observed with ELC.

These intracellular localization were confirmed on immunoelectron microscopy.

Two kinds of cell lines were subjected to electron microscopical analysis: —1) human Louckes B cell line treated with 1 μg of NPC serum-derived LMP1/exosome complex and —2) human Raji B cell line treated only with S12.

Louckes cells were treated for 48 hours with exosome/LMP-1 complex purified from NPC. Cell pellets were cutted in frozen state, then placed on slide.

CD63 was detected by anti-CD63 coupled with 10 nm gold bead. LMP-1 was detected by S12 coupled with 5 nm gold bead.

Raji cells were treated with S12 antibody for 48 hours, then fixed. The slides were treated either anti-mouse Ig (for S12) or anti-CD63 (for exosome). Anti-mouse Ig (coupled with 5 nm gold bead) reacted to S12 antibody localizing on exosome/LMP-1/S12 complex and anti-CD63 (coupled with 10 nm gold bead) for CD63 localizing on the same exosome. Positive response in exosomal vesicule, multi-vesicule, cavity and nuclei.

Exosome/LMP-1 complex isolated from serum of NPC patient has a powerful mitogenic activity on MTT test (Houali K, X. Wang, Y. Shimizu, D. Djennaoui, J. Nicholls, S. Fiorini, A. Bougermouh and T. Ooka. Clin. Cancer Res. 13: 4993-5000).

A comparative study was done on diverse cell lines in examining whether exosome/LMP-1 complex from serum of NPC patients (ELC) and exosome from serum of normal individuals (EC) have a mitogenic activity. AKATA (EBV⁻ variant), Louckes (B cell), CEM-1 (T cell), Balb/c3T3 (rodent fibroblast) and EBV-negative human epithelial HaCaT cell lines were subjected to the examination (FIG. 3).

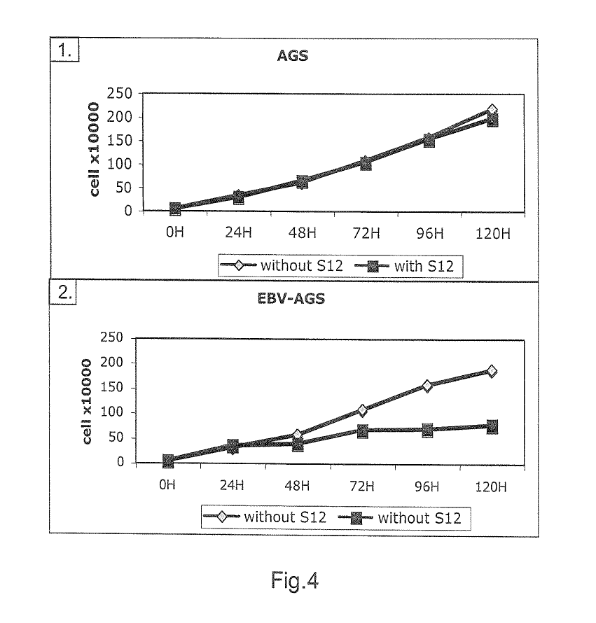

MTT test was carried out 50 000 cells/100 μl of culture medium (without FCS) with 300 ng of exosome/LMP-1 complex purified from NPC patient (SNPC). With or without FBS and exosome isolated from healthy individuals (EC-SNF) were used as controls (FIG. 4).

Exosome/LMP-1 complex from NPC showed a powerful mitogenic activity. The value obtained with ECL(SNPC) was comparable to those obtained with FBS, while PBS and EC(SNP) from healthy individuals showed a basal value. Mitogentic activation obtained with ELC(SNPC) come from the presence of LMP-1 in exosome, because addition of S12 in exosome/LMP-1 assay abolished almost totality of mitogenic activity induced with exosome/LMP-1 complex (ELC+S12) (FIG. 4, ELC(SNPC)+S12).

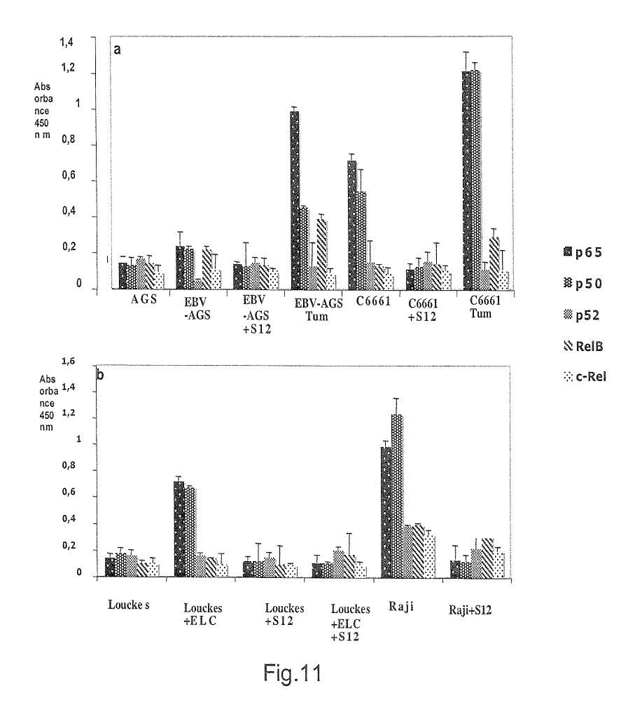

Cell death induced by anti-LMP-1 would associate with inhibition of NFkB expression, in particular two major component of NFkB (p65 and p50) (see FIG. 11) by LMP-1 complexed with exosomes. Free LMP-1 (without exosome) could not activate cell cycle (Houali K, X. Wang, Y. Shimizu, D. Djennaoui, J. Nicholls, S. Fiorini, A. Bouguermouh and T. Ooka. Clin. Cancer Res. 2007. 13: 4993-5000). Furthermore, our data showed that exosome purified from healthy individuals (EC:SNP) was unable to activate cell cycle (FIG. 4).

In conclusion, the mitogenic activity of LMP-1 requires its association with exosome. Finally, our data demonstrated that exosome/LMP-1-complexed form was capable of inhibiting NFkB expression.

NFkB expression was totally inhibited in S12-treated c666-1 and S12-treated Raji cells (FIG. 11).

Our data suggest that the inhibition of NFkB by LMP-1 reported so far in the literatures come probably from its complex with exosomes entering into cells. This observation would offer us a new concept on the oncogenic mechanism induced by LMP-1.

Effect of anti-LMP-1 was studied in EBV-AGS cell line. AGS and EBV-AGS cell lines were treated by with anti-LMP-1 (FIG. 4-1 and FIG. 4-2).

Anti-LMP-1 did not show any inhibition on AGS cell growth (FIG. 4-1), while anti-LMP-1 stoped cell growth over at 72 hours. All cells are however viable til 120 hours (FIG. 4-2).

In vitro-cultured EBV-AGS, LMP-1 transcription was almost negative. Anti-LMP-1 is therefore not toxic in these cells.

However, actually there is no explanation about the inhibition of cell growth without cell death.

### In Vivo Experiment:

We investigated the activity of anti-LMP-1 antibody in nude mice injected subcutaneously with 10⁷ cultured cells from EBV-associated tumors: c666-1 cells (derived from NPC) or EBV-positive AGS (derived from GC).

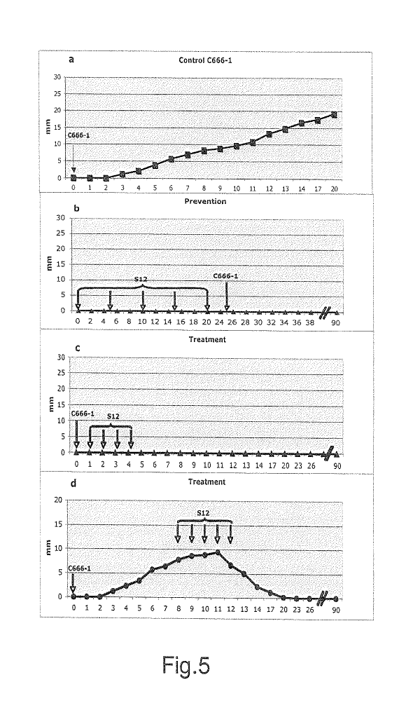

With c666-1 cells, tumor is detectable in untreated mice by the second or third day, reaches a diameter of ca. 2 mm by day 4, and 8 mm at day 8, then 16 mm at day 14 and 20 mm at 20 days (FIG. 5-1): about 1.5 folds more with EBV-AGS cell than those with c666-1 cell.

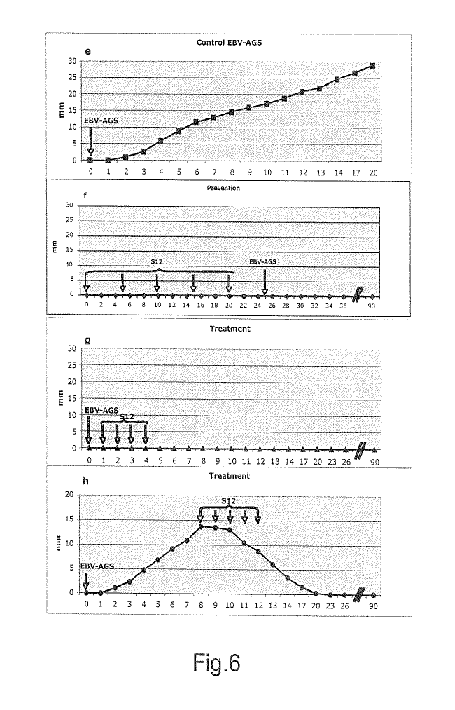

With EBV-positive AGS cells, tumor is detectable in untreated mice by the second or third day, reaches a diameter of ca. 3 mm by day 4, and 15 mm at day 8, then 25 mm at day 14 and 30 mm at 20 days (FIG. 6-1).

Induced tumors (tumor size in mm in diameter) are slightly larger with EBV-AGS cells than with c666-1 cells, about 1.5 folds (FIG. 5-a and FIG. 6-e).

To analyse the effect of anti-LMP-1, 25 μg of monoclonal anti-LMP-1 S12 per mice was injected by intraperitoneal way in three protocols:

Protocol #1, anti-LMP-1 S12 was administered as 5 intraperitoneal injections of 25 μg at 5 day intervals finishing 3 days before tumor challenge in the preventive protocol (FIG. 5-b for c666-1 and FIG. 6-f for EBV-AGS)

Protocol #2, 5 successive daily injections starting either simultaneously with tumor challenge (FIG. 5-c for c666-1 and FIG. 6-g for EBV-AGS).

Protocol #3, 5 injections (one injection everyday) when the tumor size became about 0.8 cm in diameter (FIG. 5-d for c6666-1 and FIG. 6-h for EBV-AGS).

Protocol #1 and #2 are for prevention and protocol #3 is tumor treatment. Preventive (protocol #1 —FIG. 5-b for c666-1 and FIG. 6-f for EBV-AGS) or simultaneous (protocol #2-FIG. 5-c for c666-1 and FIG. 6-g for EBV-AGS) treatment with anti-LMP-1 for both cell lines completely abrogated tumor appearance in any of the treated mice for at least 3 months.

Injection of anti-LMP-1 antibody was also highly effective if given when the tumors had already reached a considerable size. Nodules of ca. 8 mm (c666-1) and ca. 15 mm (EBV-AGS) rapidly stabilized, then regressed progressively after treatment by 5 daily injections of anti-LMP-1 antibody (FIG. 5-d for c666-1 and FIG. 6-h for EBV-AGS). The tumor masses disappeared completely at 11 days after onset of treatment, and the mice remained tumor-free for at least 3 months.

To confirm the specificity of anti-LMP-1 on the inhibition of tumor growth, we injected either EBV-encoded DNAase antibody or mouse monolonal anti-Ig antibody in Protocol #1(Preventive). Either anti-EBV-DNAase or anti-mouse Ig was administered as 5 intraperitoneal injections of 25 μg at 5 day intervals finishing 3 days before tumor challenge in the preventive protocol.

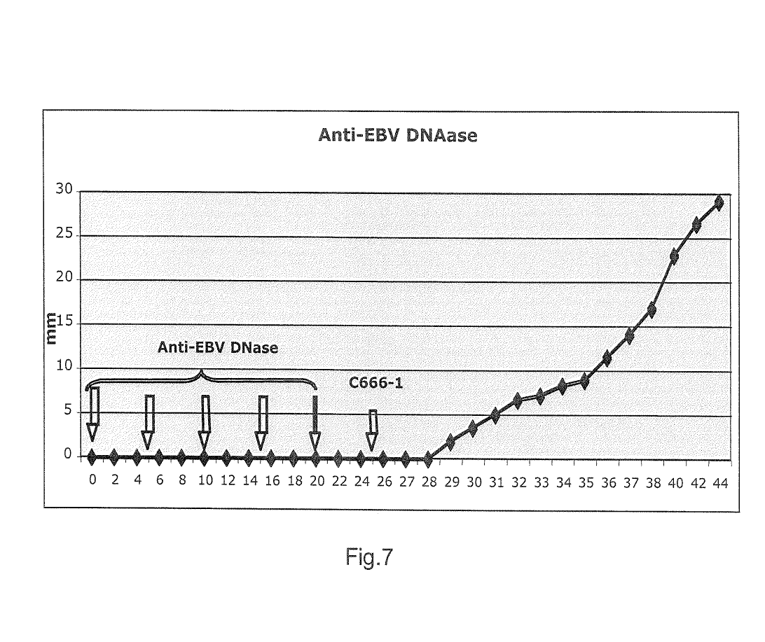

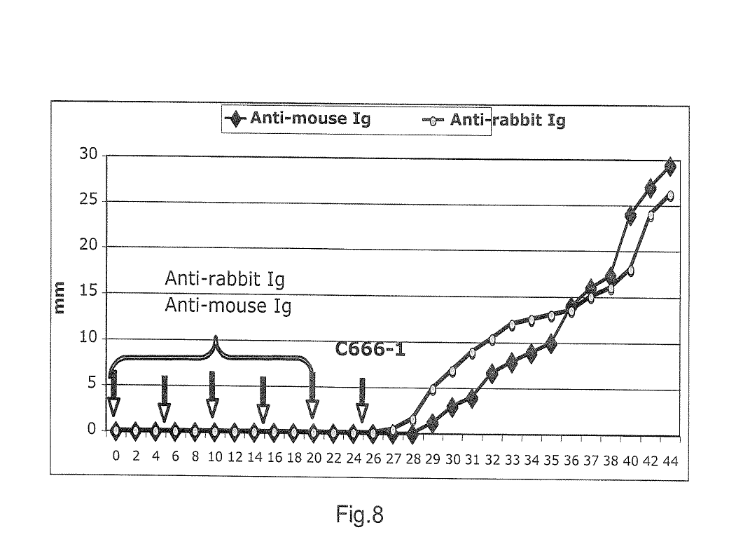

When untreated or treated animals with anti-DNAase in protocol #1 (preventive) with c666-1 (FIG. 7) or with anti-mouse Ig (FIG. 8) in the place of anti-LMP-1 used as control experiment showed rapid tumor growth (FIG. 7 and FIG. 8). This suggests that specific inhibition of tumor development is probably due to neutralisation of LMP-1 protein by S12 anti-LMP-1. Anti-mouse Ig was purchased from Sigma (France) Cat. N^{o} 62197.

Rabbit polyclonal anti-DNAase used here was produced in our laboratory from EBV-DNAase obtained by Baculovirus system (Sbih-Lammali F, Berger F, Busson P and Ooka T, 1996, Virology, 222: 64-74) (Zeng Y, Middeldorp J, Madjar J J and Ooka T, 1997, Virology 239:285-295).

We then examined if the complex of anti-LMP-1 and LMP-1 protein is present in serum as well as in tumor cells.

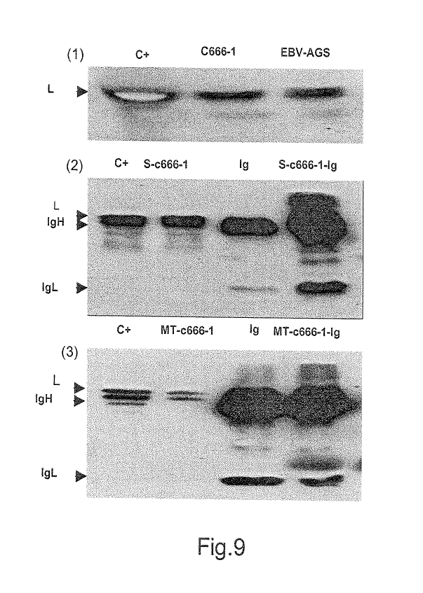

We analysed serum and tumor from tumor developping mice by immunoblot method. LMP-1 was present in the serum of mice bearing c666-1 (FIG. 9-1, c666-1) and EBV-AGS (FIG. 9-1. EBV-AGS). Positive control used in this experiment come from cellular extract of human P3HR1 B cell. LMP-1 protein was detected as classically known p63 kDa protein.

We then investigated these serum components in mice treated with antibody after the development of tumor (protocol #3). LMP-1 complexed with exosome was purified by ultracentrifugation (Houali K, X. Wang, Y. Shimizu, D. Djennaoui, J. Nicholls, S. Fiorini, A. Bouguermouh and T. Ooka. Clin. Cancer Res. 13: 4993-5000).

Analysis of complex in serum of c666-1-treated mice by Western blot shows the presence of LMP-1 (FIG. 9-2: S-c666-1) associated with rabbit immunoglobulin (FIG. 9-2: S-c666-1-Ig). Commercial mouse Ig was added as a control positive (FIG. 9-2: Ig).

Similar complexes were also present in tumor biopsies (FIG. 9-3, MT-c666-1) in association with rabbit immunoglobulin (FIG. 9-3, MT-c666-1-Ig). Commercial mouse Ig was added as a control positive (FIG. 9-3: Ig).

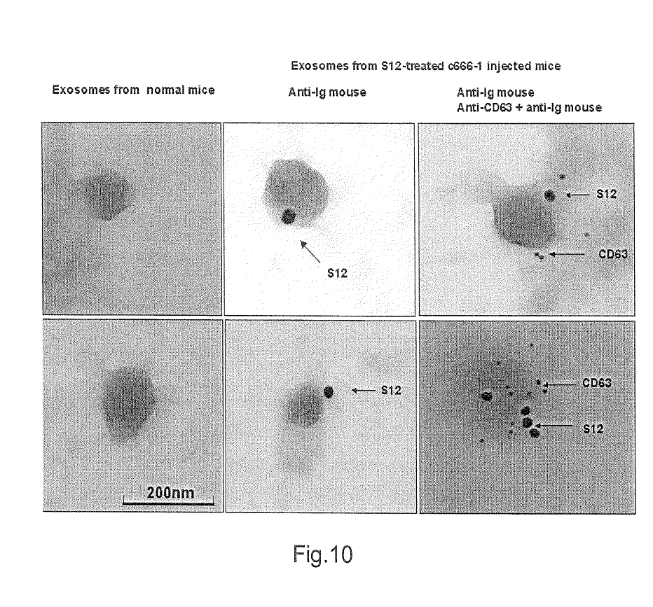

The presence of exosome/LMP-1/mouse Ig complex was searched in serum of S12-treated mice developping c666-1 tumor (FIG. 10).

Exosome/LMP-1/S12 complex from mouse serum developping c666-1 tumor was purified by differential ultracentrifugation and treated with anti-mouse Ig (for detection of S12) or anti-CD63 (for detection of exosome). Detection of exosome/LMP-1/S12 complex by 10 nm glod-labeled mouse Ig and by 5 nm gold-labeled anti-CD63. Normal exosome:exosome/LMP-1/S12 not-treated by these antibodies (anti-mouse Ig and anti-CD63 (FIG. 10) exosomes from 12-treated c666-1 injected mice).

Immunological specificity was controlled by the omission of primary antibodies or their replacement by non-immune serum (exosome from normal mice).

To visualize more precisely exosome/LMP-1/mouse Ig complex, this complex was searched on c666-1 and EBV-AGS tumor cells extracted from tumoral biopsy layered out on slide and fixed with aceton.

Surprisingly, we found the exosome/LMP-1/mouse Ig complexes inside of cells isolated from tumor biopsy from the appropriately treated mice. The complex was revealed by anti-mouse Ig for S12. In both tumors (LMP-1 c666-1 and LMP-1 EBV-AGS), exosome/LMP-1/mouse Ig complexes were seen as intracytoplasmic and intranuclear patches. Apparently, these usually mitogenic components were rendered ineffective through combination with its specific antibodies.

Complexes obtained from the sera of mice treated with S12 antibody reacted with both anti-mouse Ig and anti-CD63, confirming the presence of LMP-1/exosome complex.

Apparently, antibody neutralizes the mitogenic activity of LMP-1/exosome complex, with subsequent cell death. It was surprising that S12 antibody suppresses tumor growth in EBV-AGS implanted mice (FIG. 6, f.g.h) although these cells do not produce detectable LMP-1 expression when cultured in vitro (Kassis J, Maeda A, Teramoto N, Takada, K, Wu C, Wells A. Int. J. Cancer 2002; 99: 644-51)(see also FIG. 9 and [0095]).

Transcription of LMP-1 was compared in EBV-AGS cells ex vivo and in culture by semi-quantitative RT-PCR. We found that LMP-1 expression (a band of 479 bp) is almost absent in EBV-AGS cell culture, while its expression became positive in tumor biopsy. As expected, amplification of genomic sequence (non-spliced sequence) gave a band of 640 bp. The sequence amplified by RT-PCR corresponds to LMP1 mRNA. We confirmed these results by quantitative RT-PCR. Relative expression was presented by percentage (%) of BARF1 mRNA/actin mRNA. Transcription level almost seven folds in c666-1 tumor (c666-1/c666-1-T) in comparison with the value obtained from cultured cells (c666-1). Remarkably high activation of BARF1 transcription was observed in EBV-AGS tumor, while almost no transcription in EBV-AGS cell in culture.

Suppressive effect of anti-LMP-1 on EBV-AGS tumor is therefore due to the activation of LMP-1 expression in tumor. These observations were never done so far.

LMP-1 activates NF-kB expression (Kieff and Rickinson, 2007, Fields Virology 5th Edition-Fields B N, Knipe D M, Howley P M (ed.) Lippincott-Williams & Wilkins Publishers: Philadelphia, 2007, pp. 2603-2654). We examined the expression of five components of NF-kB by ELISA test (TransAM NFkB family Kit: Ref. 43296, Active-Motif, France). We found that treatment with S12 antibody completely suppressed NF-kB p65 and p50, ones of important components of NF-kB family in Raji and c666-1 cells from 24 hours post treatment (FIG. 11 a: c666-1+S12 and Raji+S12), suggesting that expression of NF-kB p65 and p50 in these cells depends entirely on activation by LMP-1. EBV-AGS and which do not express LMP-1, continued to show a basal expression of NF-kB p65 and p50 after treatment with S12 (FIG. 11 a), suggesting an alternative activation pathway. The p65 and p50 were also activated significantly in both type of tumor (NPC:c666-1Tum and GC: EBV-AGSTum). Activation of these components were observed when Louckes cell was treated in vitro with LMP1/exosome complex isolated from NPC serum (Louckes+ELC) (FIG. 11 b). This activation was totally reduced by the presence of S12 antibody suggesting that the activation was due to the presence of LMP 1 complexed with exosome (Louckes+ELC+S12). As positive control, significant expression of p65 and p50 in Raji cells (Raji) was also totally inhibited by S12 antibody (FIG. 11 b: Raji+S12). Treatment and prevention based on immunotherapy by anti-LMP-1 is efficient not only for NPC type carcinoma, but also GC type carcinoma Inhibitory effect by anti-LMP1 was observed in vivo and in vitro.

1. A method for preventing or treating Epstein-Barr Virus positive tumors comprising administering to patient in need thereof an effective amount of an antibody or an antibody fragment binding specifically to the polypeptide derived from Epstein-Barr Virus protein LMP1 having the sequence from position 188 to position 386 of SEQ ID No. 1.

2. A method according to claim 1 wherein the antibody or an antibody fragment binds specifically to a fragment of at least 10 amino acids of the polypeptide derived from Epstein-Barr Virus protein LMP1 having the sequence from position 188 to position 386 of SEQ ID No. 1.

3. A method according to claim 1 wherein the antibody or antibody fragment binds specifically to the polypeptide derived from Epstein-Barr Virus protein LMP1 having the sequence from position 306 to position 318 of SEQ ID No. 1.

4. A method for preventing or treating Epstein-Barr Virus positive tumors comprising administering to a patient in need thereof an effective amount of a fragment of at least 10 amino acids of the polypeptide derived from Epstein-Barr Virus protein LMP1 having the sequence from position 188 to position 386 of SEQ ID No. 1.

5. A method according to claim 4 wherein the fragment comprises the polypeptide derived from Epstein-Barr Virus protein LMP1 having the sequence from position 188 to position 386 of SEQ ID No. 1.

6. A method according to claim 4 wherein the fragment comprises the polypeptide derived from Epstein-Barr Virus protein LMP1 having the sequence from position 306 to position 318 of SEQ ID No. 1.

7. A method for preventing or treating Epstein-Barr Virus positive tumors comprising administering to a patient in need thereof an effective amount of a polynucleotide encoding a polypeptide selected from the group consisting of: a fragment of at least 10 amino acids of the polypeptide derived from Epstein-Barr Virus protein LMP1 having the sequence from position 188 to position 386 of SEQ ID No. 1, a polypeptide derived from Epstein-Barr Virus protein LMP1 having the sequence from position 188 to position 386 of SEQ ID No. 1 and a polypeptide derived from Epstein-Barr Virus protein LMP1 having the sequence from position 306 to position 318 of SEQ ID No. 1.

8. (canceled)

9. (canceled)

10. (canceled)

11. Peptide derived from Epstein-Barr Virus protein LMP1 selected from the group consisting of:
a peptide having the sequence from position 306 to position 318 of SEQ ID No. 1,
a fragment of at least 5 amino acids of a peptide having the sequence from position 306 to position 318 of SEQ ID No. 1.

12. Polynucleotide encoding a peptide according to claim 11.

13. Host cell transformed with a polynucleotide according to claim 12.

14. A method according to claim 1, wherein the Epstein-Barr Virus positive tumor is selected from the group consisting of nasopharyngeal carcinoma, gastric carcinoma, Burkitt's lymphoma, Hodgkin's lymphoma, lymphoma induced in AIDS patients, esophage and intrahepatic cholangiocarcinoma, nasal NK/T-cell lymphoma and oral hairy leucoplasia (OHL).

15. A method according to claim 4, wherein the Epstein-Barr Virus positive tumor is selected from the group consisting of nasopharyngeal carcinoma, gastric carcinoma, Burkitt's lymphoma, Hodgkin's lymphoma, lymphoma induced in AIDS patients, esophage and intrahepatic cholangiocarcinoma, nasal NK/T-cell lymphoma and oral hairy leucoplasia (OHL).

16. A method according to claim 7, wherein the Epstein-Barr Virus positive tumor is selected from the group consisting of nasopharyngeal carcinoma, gastric carcinoma, Burkitt's lymphoma, Hodgkin's lymphoma, lymphoma induced in AIDS patients, esophage and intrahepatic cholangiocarcinoma, nasal NK/T-cell lymphoma and oral hairy leucoplasia (OHL).

17. A composition comprising an antibody or an antibody fragment binding specifically to the polypeptide derived from Epstein-Barr Virus protein LMP1 having the sequence from position 188 to position 386 of SEQ ID No. 1.


SEQUENCE LISTING (1 sequence)

SEQ ID NO: 1 - protein, 386 aa, Human Herpesvirus 4 (HHV-4)
MEHDLERGPPGPRRPPRGPPLSSSLGLALLLLLLALLFWLYIVMSDWTGGALLVLYSFALMLIIIILIIFIFRRDLLCPLGALCILLLMITLLLIALWNLHGQALFLGIVLFIFGCLLVLGIWIYLLEMLWRLGATIWQLLAFFLAFFLDLILLIIALYLQQNWWTLLVDLLWLLLFLAILIWMYYHGQRHSDEHHHDDSLPHPQQATDDSGHESDSNSNEGRHHLLVSGAGDGPPLCSQNLGAPGGGPDNGPQDPDNTDDNGPQDPDNTDDNGPHDPLPQDPDNTDDNGPQDPDNTDDNGPHDPLPHSPSDSAGNDGGPPQLTEEVENKGGDQGPPLMTDGGGGHSHDSGHGGGDPHLPTLLLGSSGSGGDDDDPHGPVQLSYYD